(19) United States
(12) Patent Application Publication
(10) Pub. No.: US 2009/0023903 A1
(43) Pub. Date: Jan. 22, 2009

(54) PROCESS FOR THE PREPARATION OF TRIFLUOROALKYL-PHENYL AND HETEROCYCLIC SULFONAMIDES

(75) Inventors: Terrence Joseph Connolly, Warwick, NY (US); Anita Wai-Yin Chan, Wayne, PA (US); Zhixian Ding, Fort Lee, NJ (US); Mousumi Ghosh, Elmwood Park, NJ (US); Xinxu Shi, Flushing, NY (US); Jianxin Ren, Nanuet, NY (US); Eric Hansen, Ridgewood, NJ (US); Roger Farr, Whitehouse Station, NJ (US); Michael MacEwan, Monroe, NY (US); Asaf Alimardanov, Nanuet, NY (US); Antonia Nikitenko, Tarrytown, NY (US); John Potoski, West Nyack, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 12/172,486

(22) Filed: Jul. 14, 2008

Related U.S. Application Data
(60) Provisional application No. 60/959,659, filed on Jul. 16, 2007.

(51) Int. Cl. C07C 311/17; C07C 311/00; C07D 333/34; C07D 263/20; C07C 33/46; C07D 265/30; C09B 29/36

(52) U.S. Cl. 534/732; 564/80; 549/65; 562/405; 548/230; 568/812; 544/176

(57) ABSTRACT

A novel trifluoroacetylating agent, i.e., N-trifluoroacetylmorpholine, is described. This reagent is useful in the preparation of phenyl and heterocyclic sulfonamide compounds. Methods are therefore described for preparing sulfonamide compounds of the following structure, wherein R¹ and R² are defined herein, using N-trifluoroacetylmorpholine. The sulfonamide compounds that may be prepared as described herein include 5-chloro-thiophene-2-sulfonic acid [(1S,2R)-2-(3,5-difluoro-phenyl)-3,3,3-trifluoro-1-hydroxymethyl-propyl]-amide using N-trifluoroacetylmorpholine.

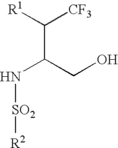

### CROSS-REFERENCE TO RELATED APPLICATIONS

This application claims the benefit of the priority of U.S. Provisional Patent Application No. 60/959,659, filed Jul. 16, 2007.

### BACKGROUND OF THE INVENTION

This invention relates to inhibitors of beta amyloid production, which have utility in the treatment of Alzheimer's disease.

Alzheimer's disease (AD) is the most common form of dementia (loss of memory) in the elderly. The main pathological lesions of AD found in the brain consist of extracellular deposits of beta amyloid protein in the form of plaques and angiopathy and intracellular neurofibrillary tangles of aggregated hyperphosphorylated tau protein. Recent evidence has revealed that elevated beta amyloid levels in the brain not only precede tau pathology but also correlate with cognitive decline. Further suggesting a causative role for beta amyloid in AD, recent studies have shown that aggregated beta amyloid is toxic to neurons in cell culture.

Heterocyclic- and phenyl-sulfonamide compounds, specifically fluoro- and trifluoroalkyl-containing heterocyclic sulfonamide compounds, have been shown to be useful for inhibiting β-amyloid production.

What are needed in the art are alternate processes for preparing sulfonamide compounds useful for inhibiting β-amyloid production.

### SUMMARY OF THE INVENTION

In one aspect, methods for preparing trifluoroalkyl-phenyl and heterocyclic sulfonamide compounds using N-trifluoroacetylmorpholine are provided.

In another aspect, methods for preparing sulfonamide compounds of the following structure, wherein R¹ and R² are defined below, using N-trifluoroacetylmorpholine are provided.

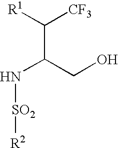

In a further aspect, methods for preparing 5-chloro-thiophene-2-sulfonic acid [(1S,2R)-2-(3,5-difluoro-phenyl)-3,3,3-trifluoro-1-hydroxymethyl-propyl]-amide using N-trifluoroacetylmorpholine are provided.

In still a further aspect, intermediates are provided which are useful in the preparation of 5-chloro-thiophene-2-sulfonic acid [(1S,2R)-2-(3,5-difluoro-phenyl)-3,3,3-trifluoro-1-hydroxymethyl-propyl]-amide.

In yet another aspect, N-trifluoroacetylmorpholine is provided.

In still a further aspect, the following compound is provided.

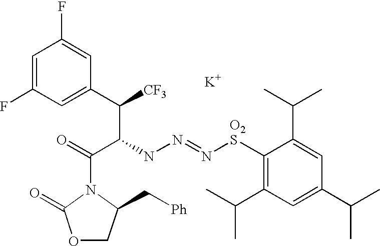

In another aspect, processes for trifluoroacetylating chemical compounds using N-trifluoroacetylmorpholine are provided.

Other aspects and advantages of the invention will be readily apparent from the following detailed description of the invention.

### BRIEF DESCRIPTION OF THE DRAWINGS

FIG. 1. provides the powder X-ray diffraction pattern for a sample of 5-chloro-thiophene-2-sulfonic acid [(1S,2R)-2-(3,5-difluoro-phenyl)-3,3,3-trifluoro-1-hydroxymethyl-propyl]-amide prepared as described herein.

### DETAILED DESCRIPTION OF THE INVENTION

A novel trifluoroacetylating reagent, i.e., N-trifluoroacetylmorpholine, is provided. Also provided are methods for preparing sulfonamide compounds, including phenyl and heterocyclic sulfonamide compounds using this novel trifluoroacetylating agent. Also described are intermediates useful in the preparation of these sulfonamide compounds.

The methods described herein may be used for the preparation of the enantiomers, diastereomers, or mixtures of heterocyclic and phenyl sulfonamide compounds. In one embodiment, the sulfonamide compound prepared using trifluoroacetylmorpholine is a phenylsulfonamide. In another embodiment, the sulfonamide compound prepared using trifluoroacetylmorpholine is a heterocyclic sulfonamide compound. In a further embodiment, the sulfonamide compound prepared using trifluoroacetylmorpholine is of the following structure.

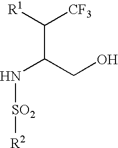

wherein, R¹ is selected from among C₁ to C₁₀ alkyl, substituted C₁ to C₁₀ alkyl, C₃ to C₈ cycloalkyl, substituted C₃ to C₈ cycloalkyl, aryl, substituted aryl, heteroaryl, and substituted heteroaryl; R² is aryl, substituted aryl, heteroaryl, or substituted heteroaryl.

In yet another embodiment, the sulfonamide compound is of the structure:

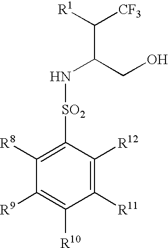

wherein, R⁸, R⁹, R¹⁰, R¹¹, and R¹² are independently selected from among H, halogen, C₁ to C₆ alkoxy, substituted C₁ to C₆ alkoxy, NO₂, C₁ to C₆ alkyl, and substituted C₁ to C₆ alkyl; or R⁸ and R⁹; R⁹ and R¹⁰; R¹¹ and R¹²; or R¹⁰ and R¹¹ are fused to form (i) a carbon-based saturated ring containing 3 to 8 carbon atoms; (ii) a carbon-based unsaturated ring containing 3 to 8 carbon atoms; or (iii) a heterocyclic ring containing 1 to 3 heteroatoms selected from among O, N, and S in the backbone of the ring; wherein rings (i) to (iii) may be substituted by 1 to 3 substituents including C₁ to C₆ alkyl or substituted C₁ to C₆ alkyl. In a further embodiment, the sulfonamide compound is of the following structure:

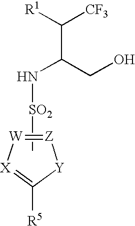

wherein, W, Y and Z are independently selected from among C, CR⁶ and N, wherein at least one of W, Y or Z is C; X is selected from among O, S, SO₂, and NR⁷; R⁵ is selected from among H, halogen, and CF₃; R⁶ is selected from among H, halogen, C₁ to C₆ alkyl, and substituted C₁ to C₆ alkyl; R⁷ is selected from among H, C₁ to C₆ alkyl, and C₃ to C₈ cycloalkyl. In still another embodiment, the sulfonamide compound prepared as described herein is 5-chloro-thiophene-2-sulfonic acid [(1S,2R)-2-(3,5-difluoro-phenyl)-3,3,3-trifluoro-1-hydroxymethyl-propyl]-amide.

The N-trifluoroacetylmorpholine reagent is prepared by the addition of trifluoroacetic anhydride to a mixture of morpholine and a base. The inventors found that this order of addition provided the best control over the exothermicity of the reaction. However, the addition of the reagents in other orders still provided the novel N-trifluoroacetylmorpholine. See, Scheme 1. One of skill in the art would readily be able to select a suitable base for use in the preparation of the novel trifluoroacetylating reagent. Desirably, the base neutralizes the trifluoroacetic acid by-product. In one embodiment, the base is a trialkylamine such as triethylamine, N-ethyldiisopropylamine, or N-methylmorpholine, among others. The product may be isolated using a variety of techniques including distillation, among others. In one embodiment, the trifluoroacetylating reagent is prepared by reacting trifluoroacetic anhydride, morpholine, and triethylamine (TEA).

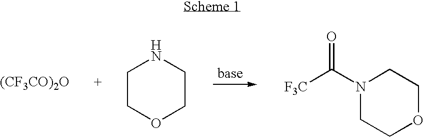

The trifluoroacetylating compound may be utilized for a variety of purposes including trifluoroacetylation.

In one aspect, the novel trifluoroacetylating agent is particularly useful in preparing sulfonamide compounds. See, Scheme 2, wherein R¹-R³ and LG are defined below.

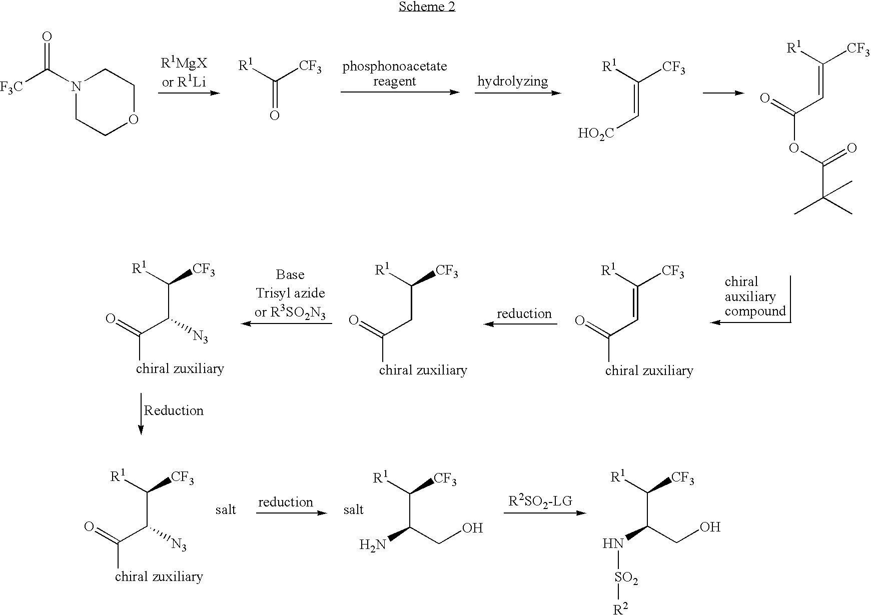

The term “alkyl” is used herein to refer to both straight- and branched-chain saturated aliphatic hydrocarbon groups. In one embodiment, an alkyl group has 1 to about 8 carbon atoms (i.e., C₁, C₂, C₃, C₄, C₅ C₆, C₇, or C₈). In another embodiment, an alkyl group has 1 to about 6 carbon atoms (i.e., C₁, C₂, C₃, C₄, C₅ or C₆). In a further embodiment, an alkyl group has 1 to about 4 carbon atoms (i.e., C₁, C₂, C₃, or C₄).

The term “cycloalkyl” is used herein to refer to cyclic, saturated aliphatic hydrocarbon groups. In one embodiment, a cycloalkyl group has 3 to about 8 carbon atoms (i.e., C₃, C₄, C₅, C₆, C₇, or C₈). In another embodiment, a cycloalkyl group has 3 to about 6 carbon atoms (i.e., C₃, C₄, C₅ or C₆).

The terms “substituted alkyl” and “substituted cycloalkyl” refer to alkyl and cycloalkyl groups, respectively, having one or more substituents including, without limitation, hydrogen, halogen, CN, OH, NO₂, amino, aryl, heterocyclic, alkoxy, aryloxy, alkylcarbonyl, alkylcarboxy, amino, and arylthio.

The term “arylthio” as used herein refers to the S(aryl) group, where the point of attachment is through the sulfur-atom and the aryl group can be substituted as noted above.

The term “alkoxy” as used herein refers to the O(alkyl) group, where the point of attachment is through the oxygen-atom and the alkyl group can be substituted as noted above.

The term “aryloxy” as used herein refers to the O(aryl) group, where the point of attachment is through the oxygen-atom and the aryl group can be substituted as noted above.

The term “alkylcarbonyl” as used herein refers to the C(O)(alkyl) group, where the point of attachment is through the carbon-atom of the carbonyl moiety and the alkyl group can be substituted as noted above.

The term “alkylcarboxy” as used herein refers to the C(O)O(alkyl) group, where the point of attachment is through the carbon-atom of the carboxy moiety and the alkyl group can be substituted as noted above.

The term “alkylamino” as used herein refers to both secondary and tertiary amines where the point of attachment is through the nitrogen-atom and the alkyl groups can be substituted as noted above. The alkyl groups can be the same or different.

The term “halogen” as used herein refers to Cl, Br, F, or I groups.

The term “aryl” as used herein refers to an aromatic, carbocyclic system, e.g., of about 6 to 14 carbon atoms, which can include a single ring or multiple aromatic rings fused or linked together where at least one part of the fused or linked rings forms the conjugated aromatic system. The aryl groups include, but are not limited to, phenyl, naphthyl, biphenyl, anthryl, tetrahydronaphthyl, phenanthryl, indene, benzonaphthyl, and fluorenyl.

The term “substituted aryl” refers to an aryl group which is substituted with one or more substituents including halogen, CN, OH, NO₂, amino, alkyl, cycloalkyl, alkenyl, alkynyl, alkoxy, C₁ to C₃ perfluoroalkyl, C₁ to C₃ perfluoroalkoxy, aryloxy, alkyloxy including —O—(C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, substituted aryl, heteroaryl, or substituted heteroaryl, which groups can be substituted. Desirably, a substituted aryl group is substituted with 1 to about 4 substituents.

The term “heterocycle” or “heterocyclic” as used herein can be used interchangeably to refer to a stable, saturated or partially unsaturated 3- to 9-membered monocyclic or multicyclic heterocyclic ring. The heterocyclic ring has in its backbone carbon atoms and one or more heteroatoms including nitrogen, oxygen, and sulfur atoms. In one embodiment, the heterocyclic ring 1 to about 4 heteroatoms in the backbone of the ring. When the heterocyclic ring contains nitrogen or sulfur atoms in the backbone of the ring, the nitrogen or sulfur atoms can be oxidized. The term “heterocycle” or “heterocyclic” also refers to multicyclic rings in which a heterocyclic ring is fused to an aryl ring of about 6 to about 14 carbon atoms. The heterocyclic ring can be attached to the aryl ring through a heteroatom or carbon atom provided the resultant heterocyclic ring structure is chemically stable. In one embodiment, the heterocyclic ring includes multicyclic systems having 2 to 5 rings.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 14-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to about 4 heteroatoms in the backbone of the ring. When the heteroaryl ring contains nitrogen or sulfur atoms in the backbone of the ring, the nitrogen or sulfur atoms can be oxidized. The term “heteroaryl” also refers to multicyclic rings in which a heteroaryl ring is fused to an aryl ring. The heteroaryl ring can be attached to the aryl ring through a heteroatom or carbon atom provided the resultant heterocyclic ring structure is chemically stable. In one embodiment, the heteroaryl ring includes multicyclic systems having 2 to 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents including halogen, CN, OH, NO₂, amino, alkyl, cycloalkyl, alkenyl, alkynyl, C₁ to C₃ perfluoroalkyl, C₁ to C₃ perfluoroalkoxy, alkoxy, aryloxy, alkyloxy including —O—(C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, substituted aryl, heteroaryl, or substituted heteroaryl which groups may be optionally substituted. A substituted heterocycle or heteroaryl group may have 1, 2, 3, or 4 substituents.

### Preparation of Sulfonamide Compounds

The first step in preparing the sulfonamide compounds includes reacting N-trifluoroacetylmorpholine with R¹MgX or R¹Li, wherein X is Br, Cl, or I and R¹ is selected from among C₁ to C₁₀ alkyl, C₁ to C₁₀ substituted alkyl, C₃ to C₈ cycloallcyl, C₃ to C₈ substituted cycloalkyl, aryl, substituted aryl, heteroaryl, and substituted heteroaryl. In one embodiment, the first step in the preparation of the sulfonamide compounds provides R¹C(O)CF₃, wherein R¹ is defined above. In another embodiment, 1-(3,5-difluoro-phenyl)-2,2,2-trifluoro-ethanone may be prepared. In one example, N-Trifluoroacetylmorpholine is reacted with 3,5-difluorophenylmagnesiumbromide to provide 1-(3,5-difluoro-phenyl)-2,2,2-trifluoro-ethanone.

The R¹C(O)CF₃ product is then condensed using a phosphonoacetate reagent. In one embodiment, the phosphonoacetate reagent is a trialkyl phosphonoacetate, wherein the alkyl groups are the same or different; triaryl phosphonoacetate wherein the aryl groups are the same or different; dialkylaryl phosphonoacetate, wherein the alkyl groups are the same or different; diarylalkylphosphonoacetate, wherein the aryl groups are the same or different; or fluoroalkyl phosphonoacetate, wherein the fluoroalkyl groups are the same or different. In another embodiment, the phosphonoacetate reagent is selected from one of the following. In a further embodiment, the phosphonoacetate reagent is triethylphosphonoacetate.

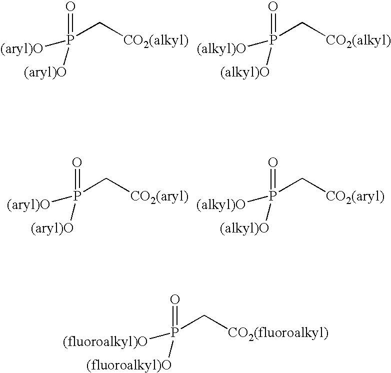

The condensation results in the production of a compound of the structure, wherein R¹ is defined above. The product may be present as a single isomer or a mixture of isomers.

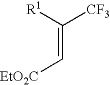

In another embodiment, the condensation results in the production of 3-(3,5-Difluoro-phenyl)-4,4,4-trifluoro-but-2-enoic acid ethyl ester. In one example, 1-(3,5-difluoro-phenyl)-2,2,2-trifluoro-ethanone is condensed using triethylphosphonoacetate to provide 3-(3,5-Difluoro-phenyl)-4,4,4-trifluoro-but-2-enoic acid ethyl ester as mixture of E-/Z-isomers, with the E-isomer being the major constituent.

The mixture is then hydrolyzed to the corresponding carboxylic acid using standard conditions and reagents known to those of skill in the art such as an aqueous base. One of skill in the art would readily be able to select a suitable aqueous base for hydrolyzing including, without limitation, an alkali metal hydroxide such as potassium hydroxide, sodium hydroxide, and lithium hydroxide. In one example, the base is an alkali metal hydroxide including KOH in a mixture of tetrahydrofuran (THF) and water.

The carboxylic acid may then be isolated by adjusting the pH of the solution to 0 to 5±0.5 (prior to addition of acid, the pH is 10-14). In one embodiment, the pH is less than about 3±0.5. Typically, an acid is utilized for the pH adjustment. One of skill in the art would readily be able to select a suitable acid for use in the pH adjustment including, e.g., hydrochloric acid, sulfuric acid, phosphoric acid, among others. Extraction may then be utilized to isolate the product by using solvents that may be readily selected by one of skill in the art. Typically, an organic solvent such as THF is utilized for the extraction. The product is then collected by reducing the solvent volume to permit precipitation of the product, which may then be collected using techniques known to those of skill in the art including filtration, centrifugation, among others. Desirably, the predominant isomer is the E-isomer. In one example, less than about 5% of the Z-isomer is present. In another example, about 1 to about 5% of the Z-isomer is present. However, one of skill in the art would recognize that the presence of the Z-isomer will not affect the production and/or isolation of the desired product. In one embodiment, R¹C(CF₃)═CHCO₂H, wherein R¹ is defined above, is prepared from the hydrolyzing. In another embodiment, E-3-(3,5-Difluoro-phenyl)-4,4,4-trifluoro-but-2-enoic acid may be prepared.

The carboxylic acid is then converted to a mixed anhydride using techniques and reagents known to those of skill in the art. Desirably, the mixed anhydride is formed via reaction between the carboxylic acid and an acid chloride. In one embodiment, the acid chloride is ClC(O)R⁴, wherein R⁴ is C₁ to C₆ alkyl. In another embodiment, the acid chloride is pivaloyl chloride. Typically, the reaction is performed in the presence of a base in an inert solvent. One of skill in the art would be able to select a suitable base in order to abstract the acidic hydrogen atom from the carboxylic acid to permit formation of the anhydride. Examples of bases that may be useful in the step include, without limitation, trialkylamines such as triethylamine, N-ethyldiisopropylamine, or N-methylmorpholine.

The term “inert solvent” as used herein is specific to the reaction being performed. Inert solvent therefore refers to a solvent that does not react or interfere with the reagents or chemical reaction therein. One of skill in the art would readily be able to select a suitable inert solvent using the teaching of the specification and particular reaction being performed therein.

One of skill in the art would therefore readily be able to select a suitable inert solvent for use in the preparation of the mixed anhydride including, without limitation, ethers or hydrocarbon solvents. In one embodiment, the inert solvent is an ether such as tetrahydrofuran. In one embodiment, the tetrahydrofuran is unsubstituted tetrahydrofuran (THF). In another embodiment, the tetrahydrofuran is 2-methyltetrahydrofuran (2-MeTHF). In one embodiment, a compound of the following structure is prepared, wherein R¹ and R⁴ are defined above:

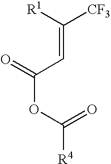

In another embodiment, a compound of the following structure is prepared, wherein R¹ is defined above.

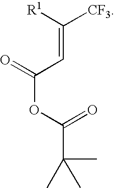

In a further embodiment, the following mixed anhydride may be prepared:

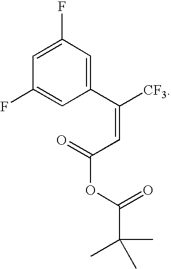

The mixed anhydride is then reacted with a chiral auxiliary compound. In one embodiment, the chiral auxiliary compound is reacted with the mixed anhydride in the presence of a strong base. The strong base utilized in this reaction must be strong enough to abstract the acidic proton from the chiral auxiliary compound to permit reaction of the chiral auxiliary compound with the mixed anhydride. Typically, the chiral auxiliary compound forms an intermediate compound such as the corresponding lithiated chiral auxiliary which then reacts with the mixed anhydride. One of skill in the art would readily be able to select a suitable strong base for use in this step including, without limitation, lithium diisopropylamine, n-hexyllithium, n-butyllithium, sec-butyllithium, lithium bis(trimethylsilylamide), sodium bis(trimethylsilylamide), or potassium bis(trimethylsilylamide. In another embodiment, the chiral auxiliary compound is reacted with the mixed anhydride in the presence of a proton scavenger. The term “proton scavenger” as used herein refers to a chemical compound that reacts with free protons in a solution. One of skill in the art would readily be able to select a suitable proton scavenger for use herein. Desirably, the proton scavenger is a trialkylamine such as triethylamine, N-ethyldiisopropylamine, or N-methylmorpholine. One of skill in the art would also understand that these reactions are performed in another inert solvent including, without limitation, THF and 2-MeTHF. In one example, the mixed anhydride is coupled with 4-(S)-Benzyl-oxazolidin-2-one in the presence of lithium diisopropylamide in THF. In another example, the mixed anhydride is coupled with 4-(S)-Benzyl-oxazolidin-2-one in the presence of lithium chloride and triethylamine.

Desirably, the chiral auxiliary compound is an oxazolidinone or an imidazolidinone. In one embodiment, the chiral auxiliary compound is selected from the following chiral auxiliaries:

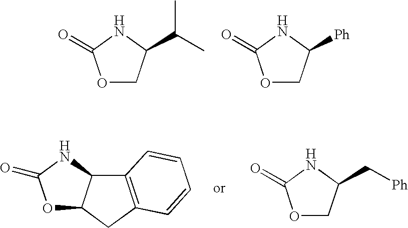

In another embodiment, the chiral auxiliary compound is an alkali metal salt of these oxazolidinones including, without limitation, the following:

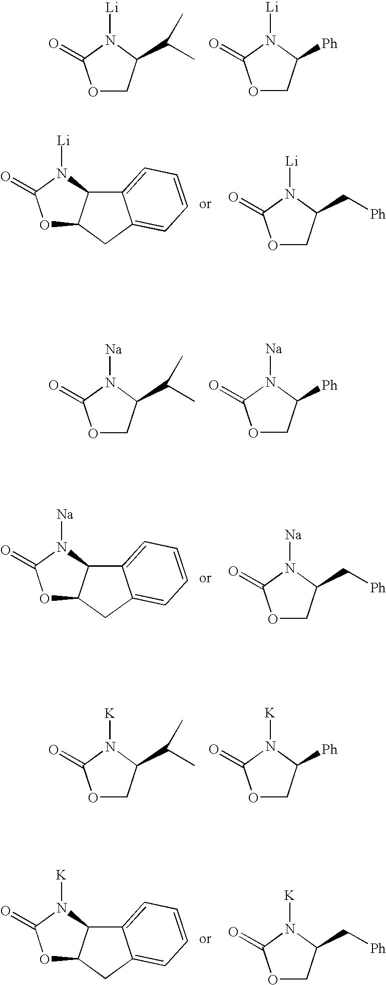

In a further embodiment, the chiral auxiliary compound is:

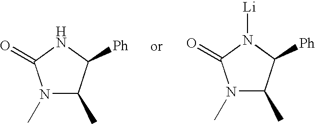

By doing so, a compound of the following structure is prepared, wherein R¹ is defined above.

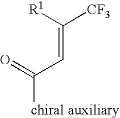

In one embodiment, a compound of the following structure is prepared, wherein R¹ is defined above. In yet another embodiment, 4-Benzyl-3-[3-(3,5-difluoro-phenyl)-4,4,4-trifluoro-but-2-enoyl]-oxazolidin-2-one is prepared.

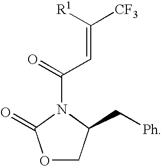

The product is then reduced using techniques and reagents known to those of skill in the art. Desirably, the reduction is performed using a Lewis Acid, a dry noble metal catalyst, and hydrogen. The term “dry” as used herein refers to the catalyst that contains less than about 5%, 4%, 3%, 2%, 1%, 0.9%, 0.8%, 0.7%, 0.6%, 0.5%, 0.4%, 0.3%, 0.2%, or 0.1% by weight water. The Lewis Acid is selected from MgCl₂ or MgBr₂, among others, and the noble metal catalyst is selected from palladium on carbon (Pd/C) or palladium on alumina (Pd/Al). In one embodiment, the Lewis acid is MgCl₂. One of skill in the art would also recognize that the reduction is performed in an inert solvent such as THF or 2-methyltetrahydrofuran. After removal of the insoluble noble metal catalyst, the reduction is then quenched using an aqueous quenching and the product is isolated using an extraction solvent such as ethyl acetate, isopropyl acetate or tert-butyl methyl ether (TBME), among others. Desirably, the extraction solvent is TBME. The product is isolated following a solvent exchange to a mixture of solvents such as isopropanol and water. One of skill in the art would be able to select suitable reagents for the quench and extraction. In one embodiment, a compound of the following structure may be prepared, wherein R¹ is defined above.

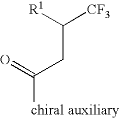

In another embodiment, a compound of the following structure may be prepared, wherein R¹ is defined above.

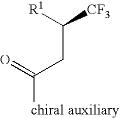

In a further embodiment, a compound of the following structure may be prepared, wherein R¹ is defined above. In yet another embodiment, (S)-4-benzyl-3-[(R)-3-(3,5-difluoro-phenyl)-4,4,4-trifluoro-butyryl]-oxazolidin-2-one is prepared from the reduction.

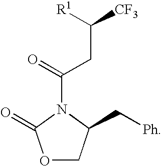

The reduced compound is then converted to its corresponding azide using trisyl azide or a sulfonyl azide such as R³SO₂N₃, wherein R³ is C₁ to C₆ alkyl, substituted C₁ to C₆ alkyl, aryl, or substituted aryl. In one embodiment, the azide is prepared using 2,4,6-triisopropylbenzenesulfonyl azide. One of skill in the art would readily be able to select a suitable agent required to prepare the azide. Desirably, the azide is prepared using reduced temperatures. In one embodiment, the azide is prepared at about 0 to about −100° C.±10° C. In another embodiment, the azide is prepared at about −40° C. to about −80° C. In a further embodiment, the azide is prepared at about −40° C. A strong base, such as lithium diisopropylamide (LDA), lithium 2,2,6,6-tetramethylpiperidide (LTMP), lithium hexamethyldisilazide (LHMDS), sodium hexamethyldisilazide (NaHMDS) or potassium hexamethyldisilazide (KHMDS) is also present. Desirably, the inert solvent is inexpensive and easy to use on a large scale. Typically, the azide is prepared in an inert solvent such as an ether, a hydrocarbon, or a mixture of these. In one embodiment, the inert solvent is an ether such as THF or 2-MeTHF. In another embodiment, the inert solvent is a hydrocarbon such as toluene.

The inventors found that when a sulfonyl azide, including the sulfonyl azides described above, are utilized to prepare the azide, the following intermediate is formed. This intermediate may then be converted to the desired azide using the quenching described below.

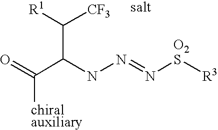

In another example, the following intermediate is preparing when using a sulfonyl azide:

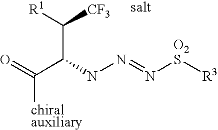

In one embodiment, the following intermediate is prepared when a sulfonyl azide is utilized to prepare the azide.

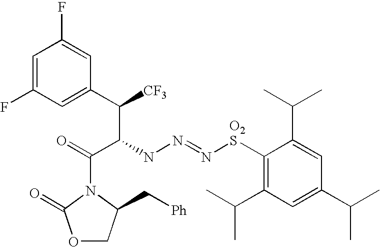

The azidation is desirably quenched using a weak acid such as a carboxylic acid. Examples of carboxylic acids useful in this quenching step include, without limitation, acetic acid, propionic acid, butyric acid, citric acid, among others. Following an aqueous work up, which may readily be performed by one of skill in the art, the azide is then isolated. In one embodiment, the solvent is exchanged with an organic solvent that provides better phase splits with aqueous systems such as methyl t-butyl ether (MTBE). In fact, the inventors found that solvent exchange not only aided in the isolation of the product, but was useful in the removal of by-products generated during the reaction. The organic solvent mixture is then washed using an aqueous base such as potassium phosphate, sodium carbonate, or potassium carbonate, among others. In another embodiment, heptane is directly added to the azidation reaction mixture and the base wash performed as described above for the organic solvent mixture. In one embodiment, an azide of the following structure may be prepared, wherein R¹ is defined above.

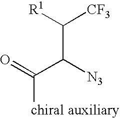

In another embodiment, an azide of the following structure may be prepared, wherein R¹ is defined above.

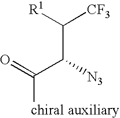

In another embodiment, an azide of the following structure may be prepared, wherein R¹ is defined above.

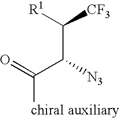

In yet another embodiment, an azide of the following structure may be prepared, wherein R¹ is defined above. In another embodiment, 3-[(S)-2-azido-(R)-3-(3,5-difluoro-phenyl)-4,4,4-trifluoro-butyryl]-(S)-4-benzyl-oxazolidin-2-one is prepared.

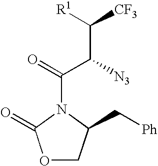

The azide is then converted to an amine salt using techniques and chemical reagents known to those of skill in the art. In one embodiment, the amine salt is prepared via a reduction using hydrogen, a noble metal catalyst such as Pd/C, and a strong acid including, without limitation, hydrochloric acid, hydrobromic acid, acetic acid, trifluoroacetic acid (TFA). Conversion of the azide to the amine salt is desirably performed in an organic solvent such as an alcohol solvent. Desirably, the alcohol solvent is ethanol, but one of skill in the art would readily be able to select another suitable organic solvent for use in this step. Upon completion of the reduction, the catalyst is removed and the amine salt is isolated using a mixture of alcohols and a non-polar hydrocarbon solvent. In one embodiment, the solvent mixture is a mixture of methanol/ethanol/heptane or ethanol-toluene. Desirably, the solvent mixture is a mixture of ethanol/heptane.

In another embodiment, the azide reduction may be performed using a Staudinger reaction. Specifically, addition of a phosphine, such as triphenylphosphine, in an inert solvent, such as THF, to the azide and a strong acid, such as concentrated HCl or HBr, provides the corresponding iminophosphorane. Hydrolyzing the iminophosphorane in the presence of water provides the corresponding amine hydrochloride or hydrobromide, respectively.

In one embodiment, the amine salt is an amine hydrochloride salt, amine hydrobromide salt, or an amine hydroacetate salt such as a hydrotrifluoroacetate salt. In a further embodiment, the amine salt is an amine hydrochloride salt. In another embodiment, the amine salt is of the structure, wherein R¹ is defined above.

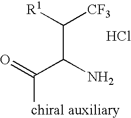

In still a further embodiment, the amine salt is of the structure, wherein R¹ is defined above.

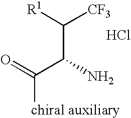

In another embodiment, the amine salt is of the structure, wherein R¹ is defined above.

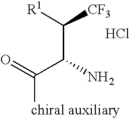

In still another embodiment, the amine salt is of the structure, wherein R1 is defined above.

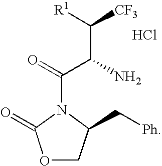

In yet a further embodiment, the amine salt is 3-[(S)-2-amino-(R)-3-(3,5-difluoro-phenyl)-4,4,4-trifluoro-butyryl]-(S)-4-benzyl-oxazolidin-2-one hydrochloride.

The chiral auxiliary group attached to the molecule is then removed to provide an amino alcohol. Desirably, the reduction is performed using a reducing agent including a metal hydride. In one embodiment, the metal hydride source is a lithium hydride such as LiBH₄, LiEt₃BH, Li(sec-Bu)₃BH, LiAlH₄, among others. One of skill in the art would readily recognize that the reaction be performed in an inert solvent such as THF, 2-MeTHF, Et₂O, toluene or a mixture thereof, among others. Because the desired amino alcohol product may originally complex with the reducing agent, a strong acid is added to quench the reduction and thereby decompose the undesired complex. One of skill in the art would readily be select a suitable strong acid for the quench. In one embodiment, the strong acid is hydrochloric acid. The amino alcohol salt is then separated from the freed chiral auxiliary group via acid/base extractions and isolated using a non-polar inert solvent. One of skill in the art would readily be able to select a suitable non-polar inert solvent for use in the extraction. In one embodiment, the non-polar inert solvent is diethyl ether, toluene or heptane or a mixture thereof. In one embodiment, an amino alcohol salt of the following structure is prepared, wherein R¹ is defined above. In another embodiment, 3-[(S)-2-Amino-(R)-3-(3,5-difluoro-phenyl)-4,4,4-trifluoro-butyryl]-(S)-4-benzyl-oxazolidin-2-one hydrochloride is the amino alcohol salt.

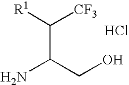

The amino alcohol salt is then coupled with R²SO₂LG, wherein R² is defined above and LG is a leaving group to provide the final sulfonamide product. The term “leaving group” as used herein refers to a chemical moiety that is easily displaced from a chemical compound. Desirably, LG is Cl, Br, imidazole or sulfonate. The coupling is performed in an organic solvent in the presence of a base, which solvent and base may readily be selected by one of skill in the art. In one embodiment, the organic solvent is methylene chloride or isopropyl acetate and the base is ethyldiisopropylamine or N-methyl morpholine. The reaction may be catalyzed by a reagent such as pyridine, 4-dimethylaminopyridine (DMAP), 4-(1-pyrrolidino)pyridine, or the N-methylpiperazine hydrochloride salt. The coupling is desirably performed at about −20 to 100° C.±5° C. In one embodiment, the coupling is performed at about 40 to 50° C. In another embodiment, the coupling is performed at about 0 to 10° C. After coupling, the sulfonamide is collected using techniques known to those of skill in the art. In one example, the crude product containing the sulfonamide is washed with a dilute acid such as aqueous HCl followed by another wash with a dilute base such as a hydroxide, carbonate, bicarbonate, phosphate and/or water and isolated as a solid from a mixture of solvent and anti-solvent such as isopropyl alcohol (IPA)/heptane, IPA/water, or dichloromethane (DCM)/heptane. In one embodiment, R²SO₂LG is 5-chlorothiophene-2-sulfonyl chloride. In another embodiment, R²SO₂LG is of the structure:

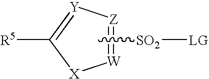

wherein, W, Y and Z are independently selected from among C, CR⁶ and N, wherein at least one of W, Y or Z is C; X is selected from among O, S, SO₂, and NR⁷; R⁵ is selected from among H, halogen, and CF₃; R⁶ is selected from among H, halogen, C₁ to C₆ alkyl, and substituted C₁ to C₆ alkyl; R⁷ is selected from among H, C₁ to C₆ alkyl, and C₃ to C₈ cycloalkyl; and point of attachment to the SO₂ group is through any one of W, Y, or Z. In a further embodiment, R²SO₂LG is of the structure:

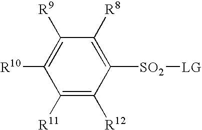

wherein, R⁸, R⁹, R¹⁰, R¹¹, and R¹² are independently selected from among H, halogen, C₁ to C₆ alkoxy, substituted C₁ to C₆ alkoxy, NO₂, C₁ to C₆ alkyl, and substituted C₁ to C₆ alkyl; or R⁸ and R⁹; R⁹ and R¹⁰; R¹¹ and R¹²; or R¹⁰ and R¹¹ are fused to form (i) a carbon-based saturated ring containing 3 to 8 carbon atoms; (ii) a carbon-based unsaturated ring containing 3 to 8 carbon atoms; or (iii) a heterocyclic ring containing 1 to 3 heteroatoms selected from among O, N, and S in the backbone of the ring; wherein rings (i) to (iii) may be substituted by 1 to 3 substituents including C₁ to C₆ alkyl or substituted C₁ to C₆ alkyl. By doing so, phenyl and heterocyclic sulfonamide compounds may be prepared.

In one example, 5-chloro-thiophene-2-sulfonic acid [(1S,2R)-2-(3,5-difluoro-phenyl)-3,3,3-trifluoro-1-hydroxymethyl-propyl]-amide may be prepared according to Scheme 3.

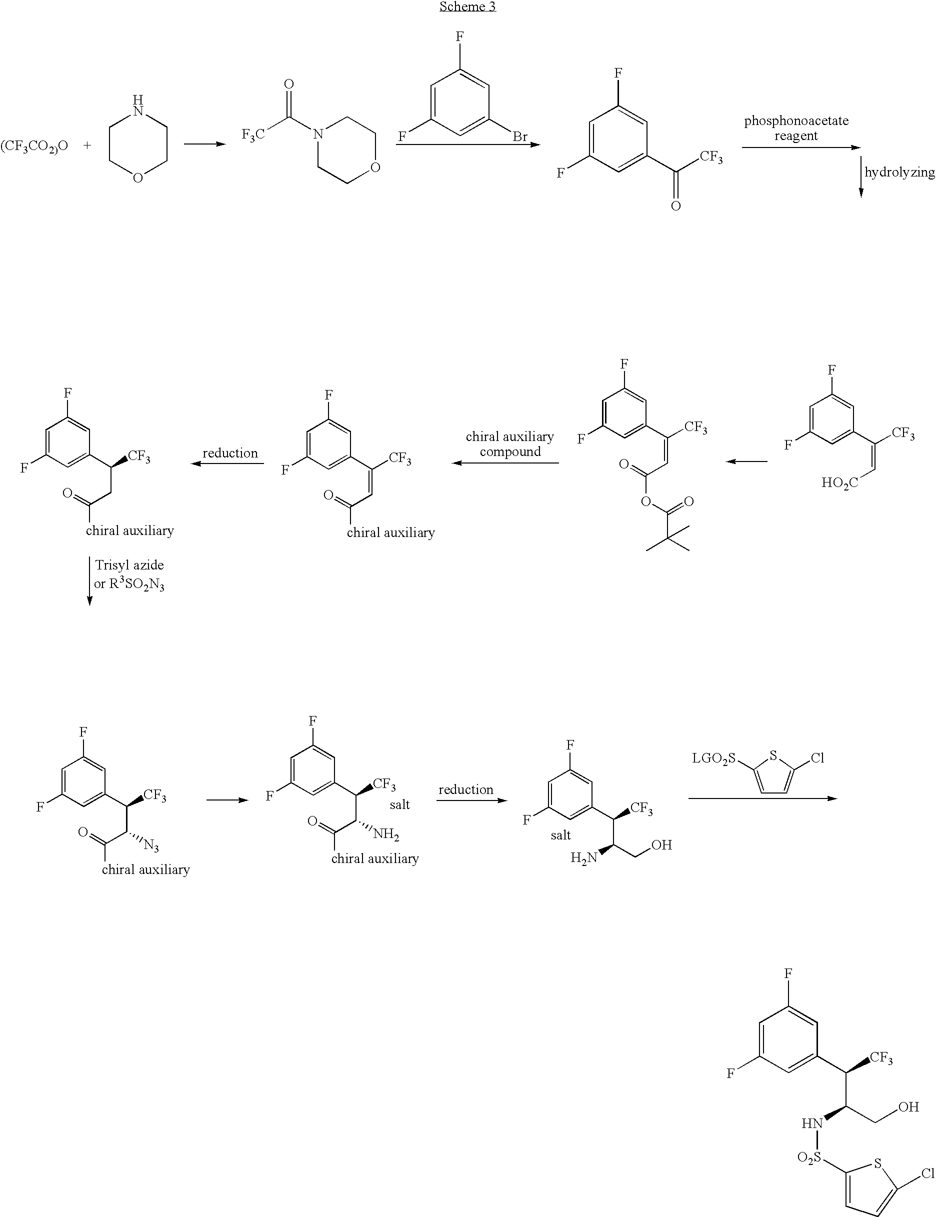

In another example, 5-chloro-thiophene-2-sulfonic acid [(1S,2R)-2-(3,5-difluoro-phenyl)-3,3,3-trifluoro-1-hydroxymethyl-propyl]-amide may be prepared according to Scheme 4.

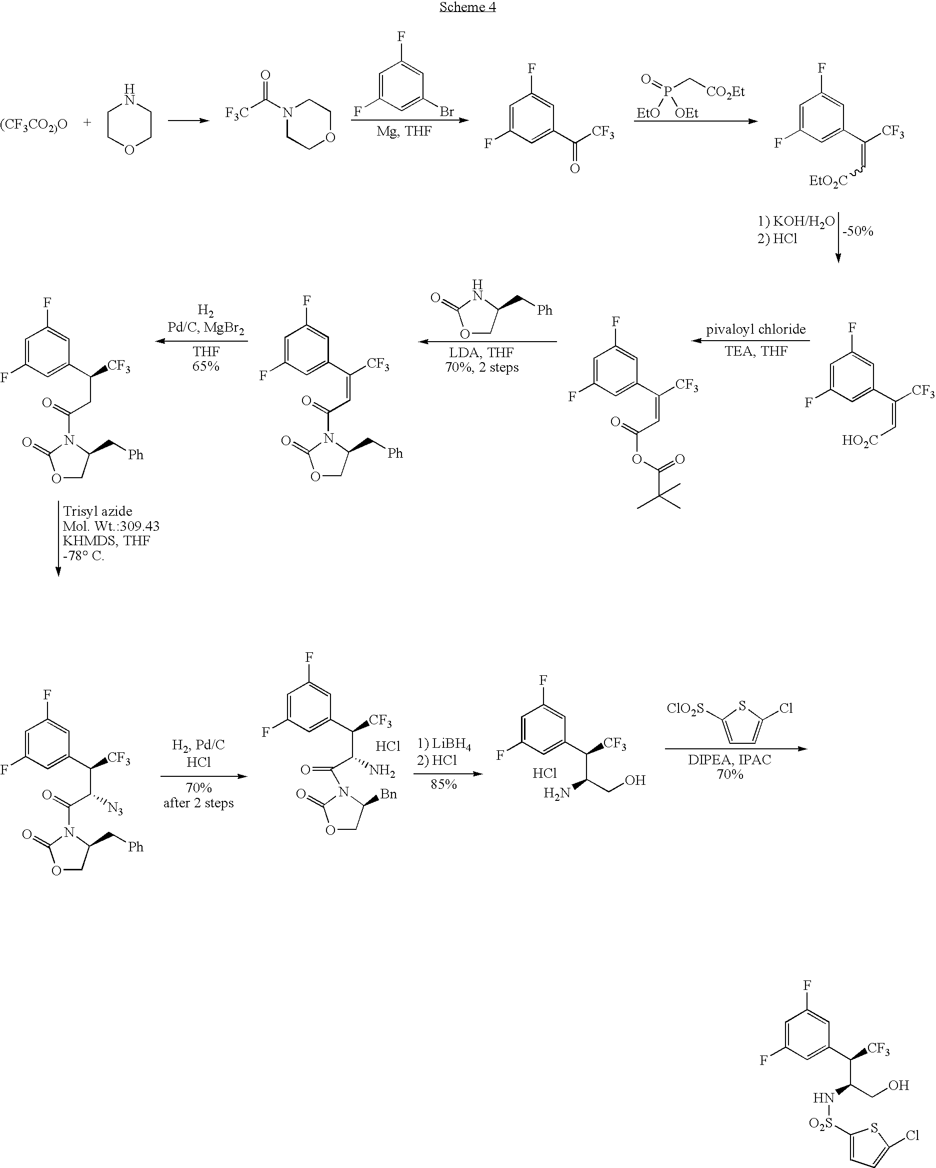

In a further example, a method is described for preparing 5-chloro-thiophene-2-sulfonic acid [(1S,2R)-2-(3,5-difluoro-phenyl)-3,3,3-trifluoro-1-hydroxymethyl-propyl]-amide, including (a) reacting N-trifluoroacetylmorpholine with bromo-3,5-difluorobenzene to form 1-(3,5-difluoro-phenyl)-2,2,2-trifluoro-ethanone; (b) reacting 1-(3,5-difluoro-phenyl)-2,2,2-trifluoro-ethanone with triethylphosphonoacetate to form 3-(3,5-difluoro-phenyl)-4,4,4-trifluoro-but-2-enoic acid ethyl ester; (c) hydrolyzing 3-(3,5-difluoro-phenyl)-4,4,4-trifluoro-but-2-enoic acid ethyl ester to form E-3-(3,5-difluoro-phenyl)-4,4,4-trifluoro-but-2-enoic acid; (d) converting E-3-(3,5-difluoro-phenyl)-4,4,4-trifluoro-but-2-enoic acid to a mixed anhydride; (e) reacting the mixed anhydride with 4-(S)-benzyl-oxazolidin-2-one to form 4-benzyl-3-[3-(3,5-difluoro-phenyl)-4,4,4-trifluoro-but-2-enoyl]-oxazolidin-2-one; (f) reducing 4-benzyl-3-[3-(3,5-difluoro-phenyl)-4,4,4-trifluoro-but-2-enoyl]-oxazolidin-2-one to form (S)-4-benzyl-3-[(R)-3-(3,5-difluoro-phenyl)-4,4,4-trifluoro-butyryl]-oxazolidin-2-one; (g) reacting (S)-4-benzyl-3-[(R)-3-(3,5-difluoro-phenyl)-4,4,4-trifluoro-butyryl]-oxazolidin-2-one with trisyl azide to form 3-[(S)-2-azido-(R)-3-(3,5-difluoro-phenyl)-4,4,4-trifluoro-butyryl]-(S)-4-benzyl-oxazolidin-2-one; (h) converting 3-[(S)-2-azido-(R)-3-(3,5-difluoro-phenyl)-4,4,4-trifluoro-butyryl]-(S)-4-benzyl-oxazolidin-2-one to 3-[(S)-2-amino-(R)-3-(3,5-difluoro-phenyl)-4,4,4-trifluoro-butyryl]-(S)-4-benzyl-oxazolidin-2-one hydrochloride; (i) reducing 3-[(S)-2-amino-(R)-3-(3,5-difluoro-phenyl)-4,4,4-trifluoro-butyryl]-(S)-4-benzyl-oxazolidin-2-one hydrochloride to (S)-2-amino-(R)-3-(3,5-difluoro-phenyl)-4,4,4-trifluoro-butan-1-ol hydrochloride; and (j) reacting (S)-2-amino-(R)-3-(3,5-difluoro-phenyl)-4,4,4-trifluoro-butan-1-ol hydrochloride with 5-chlorothiophene-2-sulfonyl chloride.

In another example, methods for preparing (E)-3-(3,5-Difluoro-phenyl)-4,4,4-trifluoro-but-2-enoic acid are described and include (a) reacting N-trifluoroacetylmorpholine with bromo-3,5-difluorobenzene to form 1-(3,5-difluoro-phenyl)-2,2,2-trifluoro-ethanone; (b) reacting 1-(3,5-difluoro-phenyl)-2,2,2-trifluoro-ethanone with triethylphosphonoacetate to form 3-(3,5-difluoro-phenyl)-4,4,4-trifluoro-but-2-enoic acid ethyl ester; and (c) hydrolyzing 3-(3,5-difluoro-phenyl)-4,4,4-trifluoro-but-2-enoic acid ethyl ester.

Iin a further example, a method is provided for preparing (S)-4-Benzyl-3-[3-(3,5-difluoro-phenyl)-4,4,4-trifluoro-but-2-enoyl]-oxazolidin-2-one and includes (a) reacting N-trifluoroacetylmorpholine with bromo-3,5-difluorobenzene to form 1-(3,5-difluoro-phenyl)-2,2,2-trifluoro-ethanone; (b) reacting 1-(3,5-difluoro-phenyl)-2,2,2-trifluoro-ethanone withtriethylphosphonoacetate to form 3-(3,5-difluoro-phenyl)-4,4,4-trifluoro-but-2-enoic acid ethyl ester; (c) hydrolyzing 3-(3,5-difluoro-phenyl)-4,4,4-trifluoro-but-2-enoic acid ethyl ester to form E-3-(3,5-difluoro-phenyl)-4,4,4-trifluoro-but-2-enoic acid; (d) converting E-3-(3,5-difluoro-phenyl)-4,4,4-trifluoro-but-2-enoic acid to a mixed anhydride; and (e) reacting the mixed anhydride with 4-(S)-benzyl-oxazolidin-2-one or a salt thereof to form 4-benzyl-3-[3-(3,5-difluoro-phenyl)-4,4,4-trifluoro-but-2-enoyl]-oxazolidin-2-one.

In yet another example, a method for preparing (S)-4-Benzyl-3-[(R)-3-(3,5-difluoro-phenyl)-4,4,4-trifluoro-butyryl]-oxazolidin-2-one is provided and includes (a) reacting N-trifluoroacetylmorpholine with bromo-3,5-difluorobenzene to form 1-(3,5-difluoro-phenyl)-2,2,2-trifluoro-ethanone; (b) reacting 1-(3,5-difluoro-phenyl)-2,2,2-trifluoro-ethanone with triethylphosphonoacetate to form 3-(3,5-difluoro-phenyl)-4,4,4-trifluoro-but-2-enoic acid ethyl ester; (c) hydrolyzing 3-(3,5-difluoro-phenyl)-4,4,4-trifluoro-but-2-enoic acid ethyl ester to form E-3-(3,5-difluoro-phenyl)-4,4,4-trifluoro-but-2-enoic acid; (d) converting E-3-(3,5-difluoro-phenyl)-4,4,4-trifluoro-but-2-enoic acid to a mixed anhydride; (e) reacting the mixed anhydride with 4-(S)-benzyl-oxazolidin-2-one or a salt thereof to form 4-benzyl-3-[3-(3,5-difluoro-phenyl)-4,4,4-trifluoro-but-2-enoyl]-oxazolidin-2-one; and (f) reducing 4-benzyl-3-[3-(3,5-difluoro-phenyl)-4,4,4-trifluoro-but-2-enoyl]-oxazolidin-2-one.

In still a further example, a method is described for preparing 3-[(S)-2-Azido-(R)-3-(3,5-difluoro-phenyl)-4,4,4-trifluoro-butyryl]-(S)-4-benzyl-oxazolidin-2-one and includes (a) reacting N-trifluoroacetylmorpholine with bromo-3,5-difluorobenzene to form 1-(3,5-difluoro-phenyl)-2,2,2-trifluoro-ethanone; (b) reacting 1-(3,5-difluoro-phenyl)-2,2,2-trifluoro-ethanone with triethylphosphonoacetate to form 3-(3,5-difluoro-phenyl)-4,4,4-trifluoro-but-2-enoic acid ethyl ester; (c) hydrolyzing 3-(3,5-difluoro-phenyl)-4,4,4-trifluoro-but-2-enoic acid ethyl ester to form E-3-(3,5-difluoro-phenyl)-4,4,4-trifluoro-but-2-enoic acid; (d) converting E-3-(3,5-difluoro-phenyl)-4,4,4-trifluoro-but-2-enoic acid to a mixed anhydride; (e) reacting the mixed anhydride with 4-(S)-benzyl-oxazolidin-2-one or a salt thereof to form 4-benzyl-3-[3-(3,5-difluoro-phenyl)-4,4,4-trifluoro-but-2-enoyl]-oxazolidin-2-one; (f) reducing 4-benzyl-3-[3-(3,5-difluoro-phenyl)-4,4,4-trifluoro-but-2-enoyl]-oxazolidin-2-one to form (S)-4-benzyl-3-[(R)-3-(3,5-difluoro-phenyl)-4,4,4-trifluoro-butyryl]-oxazolidin-2-one; and (g) reacting (S)-4-benzyl-3-[(R)-3-(3,5-difluoro-phenyl)-4,4,4-trifluoro-butyryl]-oxazolidin-2-one with trisyl azide.

In another example, a method for preparing 3-[(S)-2-Amino-(R)-3-(3,5-difluoro-phenyl)-4,4,4-trifluoro-butyryl]-(S)-4-benzyl-oxazolidin-2-one hydrochloride is provided and includes (a) N-trifluoroacetylmorpholine with bromo-3,5-difluorobenzene to form 1-(3,5-difluoro-phenyl)-2,2,2-trifluoro-ethanone; (b) reacting 1-(3,5-difluoro-phenyl)-2,2,2-trifluoro-ethanone with triethylphosphonoacetate to form 3-(3,5-difluoro-phenyl)-4,4,4-trifluoro-but-2-enoic acid ethyl ester; (c) hydrolyzing 3-(3,5-difluoro-phenyl)-4,4,4-trifluoro-but-2-enoic acid ethyl ester to form E-3-(3,5-difluoro-phenyl)-4,4,4-trifluoro-but-2-enoic acid; (d) converting E-3-(3,5-difluoro-phenyl)-4,4,4-trifluoro-but-2-enoic acid to a mixed anhydride; (e) reacting the mixed anhydride with 4-(S)-benzyl-oxazolidin-2-one or a salt thereof to form 4-benzyl-3-[3-(3,5-difluoro-phenyl)-4,4,4-trifluoro-but-2-enoyl]-oxazolidin-2-one; (f) reducing 4-benzyl-3-[3-(3,5-difluoro-phenyl)-4,4,4-trifluoro-but-2-enoyl]-oxazolidin-2-one to form (S)-4-benzyl-3-[(R)-3-(3,5-difluoro-phenyl)-4,4,4-trifluoro-butyryl]-oxazolidin-2-one; (g) reacting (S)-4-benzyl-3-[(R)-3-(3,5-difluoro-phenyl)-4,4,4-trifluoro-butyryl]-oxazolidin-2-one with trisyl azide to form 3-[(S)-2-azido-(R)-3-(3,5-difluoro-phenyl)-4,4,4-trifluoro-butyryl]-(S)-4-benzyl-oxazolidin-2-one; and (h) reacting 3-[(S)-2-azido-(R)-3-(3,5-difluoro-phenyl)-4,4,4-trifluoro-butyryl]-(S)-4-benzyl-oxazolidin-2-one with hydrogen and hydrochloric acid.

In yet another example, a method is provided for preparing (S)-2-Amino-(R)-3-(3,5-difluoro-phenyl)-4,4,4-trifluoro-butan-1-ol hydrochloride and includes (a) reacting N-trifluoroacetylmorpholine with bromo-3,5-difluorobenzene to form 1-(3,5-difluoro-phenyl)-2,2,2-trifluoro-ethanone; (b) reacting 1-(3,5-difluoro-phenyl)-2,2,2-trifluoro-ethanone with triethylphosphonoacetate to form 3-(3,5-difluoro-phenyl)-4,4,4-trifluoro-but-2-enoic acid ethyl ester; (c) hydrolyzing 3-(3,5-difluoro-phenyl)-4,4,4-trifluoro-but-2-enoic acid ethyl ester to form E-3-(3,5-difluoro-phenyl)-4,4,4-trifluoro-but-2-enoic acid; (d) converting E-3-(3,5-difluoro-phenyl)-4,4,4-trifluoro-but-2-enoic acid to a mixed anhydride; (e) reacting the mixed anhydride with 4-(S)-benzyl-oxazolidin-2-one to form 4-benzyl-3-[3-(3,5-difluoro-phenyl)-4,4,4-trifluoro-but-2-enoyl]-oxazolidin-2-one; (f) reducing 4-benzyl-3-[3-(3,5-difluoro-phenyl)-4,4,4-trifluoro-but-2-enoyl]-oxazolidin-2-one to form (S)-4-benzyl-3-[(R)-3-(3,5-difluoro-phenyl)-4,4,4-trifluoro-butyryl]-oxazolidin-2-one; (g) reacting (S)-4-benzyl-3-[(R)-3-(3,5-difluoro-phenyl)-4,4,4-trifluoro-butyryl]-oxazolidin-2-one with trisyl azide to form 3-[(S)-2-azido-(R)-3-(3,5-difluoro-phenyl)-4,4,4-trifluoro-butyryl]-(S)-4-benzyl-oxazolidin-2-one; (h) reacting 3-[(S)-2-azido-(R)-3-(3,5-difluoro-phenyl)-4,4,4-trifluoro-butyryl]-(S)-4-benzyl-oxazolidin-2-one with hydrogen and hydrochloric acid to form 3-[(S)-2-amino-(R)-3-(3,5-difluoro-phenyl)-4,4,4-trifluoro-butyryl]3-(3S-4-benzyl-oxazolidin-2-one hydrochloride; and (i) reducing 3-[(S)-2-amino-(R)-3-(3,5-difluoro-phenyl)-4,4,4-trifluoro-butyryl]-(S)-4-benzyl-oxazolidin-2-one hydrochloride.

Analysis of Compounds Prepared using the Described Methods

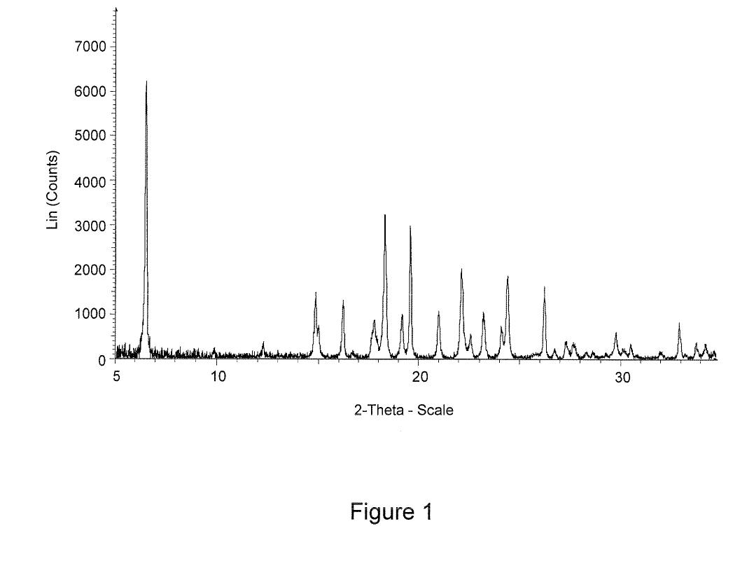

The powder XRD pattern of 5-chloro-thiophene-2-sulfonic acid [(1S,2R)-2-(3,5-difluoro-phenyl)-3,3,3-trifluoro-1-hydroxymethyl-propyl]-amide described herein was obtained using X-ray crystallographic techniques known to those of skill in the art. See, FIG. 1. In one embodiment, the XRD pattern of 5-chloro-thiophene-2-sulfonic acid [(1S,2R)-2-(3,5-difluoro-phenyl)-3,3,3-trifluoro-1-hydroxymethyl-propyl]-amide contains one large peak and several smaller peaks. The XRD for 5-chloro-thiophene-2-sulfonic acid [(1S,2R)-2-(3,5-difluoro-phenyl)-3,3,3-trifluoro-1-hydroxymethyl-propyl]-amide includes a peak at 20 of about 6.50±0.3°. The XRD for 5-chloro-thiophene-2-sulfonic acid [(1S,2R)-2-(3,5-difluoro-phenyl)-3,3,3-trifluoro-1-hydroxymethyl-propyl]-amide may also include peaks at 20 of about 14.9°±0.3°, 22.1°±0.3°, 18.3°±0.3°, 19.6°±0.3°, 24.4°+0.3°, or 26.2°±0.3° at varying intensities. One of skill in the art would readily recognize that the intensities of the peaks of the powder X-ray diffraction pattern may vary. In one embodiment, the intensities of one or more peaks of the powder X-ray diffraction pattern may vary due to crystal shape, crystal size, among others.

The following examples are illustrative only and are not intended to be a limitation on the present invention.

### EXAMPLES

### Example 1

### Preparation of N-Trifluoroacetylmorpholine

Morpholine (600 g, 6.9 mol) and triethylamine (697 g, 6.9 mol, 1 eq) were combined in a 3-L vessel equipped with overhead stirring, a thermocouple, an addition funnel and a nitrogen inlet. Trifluoroacetic anhydride (1519 g, 7.2 mol, 1.05 eq) was added while the temperature was maintained between 25 and 60° C. At the end of the addition, the reaction vessel was setup for vacuum distillation and the product collected at a head temperature of 60-80° C. (10 torr). Total yield 735.9 g (94%).

### Example 2

### Preparation of (E)-3-(3,5-Difluoro-phenyl)-4,4,4-trifluoro-but-2-enoic acid

### Method 1:

Magnesium (53.3 g) and tetrahydrofuran (0.20 L) were added to a 5-L four-necked flask equipped with an overhead stirrer, an addition funnel and a thermocouple. 3-bromo-1,5-difluorobenzene (25 g) was added to the flask and stirred until an exothermic reaction was observed. Tetrahydrofuran (1.40 L) was then added to the flask. 3-bromo-1,5-difluorobenzene (380 g) was then slowly added from the addition funnel to the flask over 2 hours, maintaining the batch temperature at 24-38° C. The reaction mixture was cooled to −26 to 5° C. and 2,2,2-Trifluoro-1-morpholin-4-yl-ethanone (306 g) was added slowly via an addition funnel over 2 hours maintaining the batch temperature at −40 to 10° C. The solution was cooled in the flask to −5 to 10° C. Triethylphosphonoacetate (402 g) was added to the reaction mixture via an addition funnel over 1 hour, while maintaining the batch temperature at 3 to 10° C. Water (1.0 L) and potassium hydroxide (615 g of a 45% aqueous solution) were added to the flask and stirred for 2 to 16 hours. Concentrated hydrochloric acid (649 g) was added to the flask, while maintaining the batch temperature at 20 to 38° C. The layers were separated and the lower aqueous phase was washed twice with tetrahydrofuran (0.80 L each). The organic layers were combined and concentrated under vacuum to a residual volume of 0.9 L. The solution was cooled to 5° C. and the resulting slurry filtered. The wet cake was dried under vacuum until the water content was less than 1.0% (KF method). The product was obtained as a yellow solid (361 g, 69% yield).

### Method 2:

Potassium carbonate (16.4 g) was suspended in ethanol (30 mL) and the solution cooled to 5 to 15° C. In a separate flask, 2,2,2,3′,5′-pentafluoroacetophenone (10 g) and triethylphosphonoacetate (10.7 g) were combined. This mixture was added to the mixture of potassium carbonate and ethanol, the temperature maintained below 25° C. The solution was stirred at ambient temperature and then cooled to 10 to 15° C. Potassium hydroxide (15 g of 40 wt % solution) was added to the solution. After completion of the hydrolysis (as monitored by HPLC), water (30 g) was added, the ethanol removed by distillation, the mixture was cooled, and 6N HCl (40 mL) was added. The precipitated product was collected, washed with water, and pulled dry (9.6 g, 80% yield).

### Example 3

### Purification of (E)-3-(3,5-Difluoro-phenyl)-4,4,4-trifluoro-but-2-enoic acid

The crude product (980 g) from Step B was added to a 5-L four-necked flask equipped with an overhead stirrer, an addition funnel and a thermocouple. Ethanol (1.5 L) was added to the flask and the mixture heated to 60° C. Water (2.0 L) was added to the flask and the mixture cooled to 5° C. The resulting slurry was stirred and then filtered on a glass funnel. The resultant cake was washed with a mixture of ethanol (0.4 L) and water (0.4 L) and the wet cake was dried under vacuum until the water content was less than 1.0% (KF method). The product was obtained as a yellow solid (782 g, 79.7%).

### Example 4

### Preparation of (S)-4-Benzyl-3-[3-(3,5-difluoro-phenyl)-4,4,4-trifluoro-but-2-enoyl]-oxazolidin-2-one

### Method 1:

THF (250 g) and triethylamine (11.1 g) were added to a 1 L reactor and the temperature adjusted to −30° C. Pivaloyl chloride (12.7 g) was added to the reactor, while maintaining the internal temperature to less than −20° C. The contents of the reactor were permitted to warm to 15 to 20° C. with agitation over 30-45 minutes, then the resulting mixed anhydride solution was cooled to −35 to −40° C.

THF (100 g) and (S)-4-benzyl-2-oxazolidinone (19.5 g) were added to another 1-L flask and the clear solution cooled to −35 to −40° C. LDA (44.7 g of a 2M in heptane, THF and ethylbenzene solution) was added and the solution maintained at an internal temperature of about −35 to −40° C. The mixture was stirred at −35 to 40° C. for a minimum of 30 minutes. The solution of (S)-benzyl-2-oxazolidinone/LDA was then transferred into the mixed anhydride reactor via a TEFLON® tube, while maintaining the temperature at −35 to −40° C. The reaction mixture was permitted to warm to 20 to 25° C. and a solution of NH₄Cl (30 g) in water (170 g) was added. The layers were separated and the upper organic layer was washed with a solution of NaCl (30 g) and water (170 g). The layers were separated and the organic layer (600 mL) was concentrated via distillation (30 mL, 1.2 parts v/w). IPA (75 g) was added to the concentrate and the suspension was heated to 60 to 70° C. Water (125 g) was added dropwise, while maintaining the temperature in the range of 60 to 70° C. The resulting cloudy solution was cooled to 20 to 25° C. over a minimum of 1 hour and the suspension stirred at 20 to 25° C. for a minimum of 30 minutes. The cloudy solution was then cooled to 0 to 5° C., the suspension filtered, the cake washed with a solution of IPA (12.5 g) and water (50 g). The filter cake was dried in a vacuum oven with a nitrogen bleed at 60° C. for a minimum of 18 hours and the product (28.1 g, 69%) collected.

### Method 2:

The carboxylic acid of step C (145.3 g), 4-(S)-benzyl-2-oxazolidinone (112 g) and LiCl (49 g) were added to the reactor. THF (1.5 Kg) was added and the mixture stirred at room temperature for 30 minutes. The resulting solution was cooled to −20 C and pivaloyl chloride (174 g) added. Triethylamine (152 g, 210 mL) was added over 30 minutes, while maintaining an internal temperature of −17° C. to −25° C. The mixture was aged at −17 C to −25° C. for an additional hour, acetic acid (33 mL) and water (500 mL) were added, and the solution mixed. The layers were separated, heptane was added to the organic layer, and the organic layer was washed with water (2×400 mL). The organic layer was concentrated on a rotovap to about 1 L and heptane (˜2 L) was added, while maintaining a batch volume of about 1 L. The crystalline slurry was cooled to −5° C. and stirred at −5° C. for 30 minutes. The crystalline solid was filtered and washed with cold (−5° C.) heptane (2×200 mL). The solid was dried in a vacuum oven and an off-white crystalline solid (212 g; 90%) was isolated.

### Example 5

### Preparation of (S)-4-Benzyl-3-[(R)-3-(3,5-difluoro-phenyl)-4,4,4-trifluoro-butyryl]-oxazolidin-2-one

MgBr₂ (580 g), Pd 10%/activated carbon (109 g) and (S)-4-Benzyl-3-[3-(3,5-difluoro-phenyl)-4,4,4-trifluoro-but-2-enoyl]-oxazolidin-2-one (1080 g) were added to a 40-L pressure reactor. The reactor was sealed and THF (19.1 kg) was added. The reactor contents were stirred and hydrogen was introduced into the reactor at a pressure of 60 to 65 psig (4.1-4.5 bar, 0.41-0.45 Mpa). The reaction mixture was stirred at 45 to 50° C., while maintaining hydrogen pressure at 50 to 65 psig (3.4-4.5 bar, 0.34-0.45 Mpa) for 4 hours. The hydrogen was vented and the reactor was flushed with nitrogen. The reaction mixture was filtered through a sparkler filter that was pre-coated with the CELITE® 503 reagent (600 g) and the cake was rinsed with MTBE (9.7 kg). The combined filtrate was washed three times with a brine solution (˜1 kg NaCl+5.5 kg water for each wash) and the organic layer was concentrated to about 8 to 10 L under vacuum (25 to 30° C. batch temperature). IPA (6 L) was added and the mixture was concentrated at 70° C. to a volume of about 4 L. Water (4 L) was added and the slurry was cooled to 25° C. The mixture was filtered and dried under vacuum at 60° C. to afford the desired product (825 g).

### Example 6

### Preparation of 3-[(S)-2-Azido-(R)-3-(3,5-difluoro-phenyl)-4,4,4-trifluoro-butyryl]-(S)-4-benzyl-oxazolidin-2-one

### Method 1:

(S)-4-Benzyl-3-[(R)-3-(3,5-difluoro-phenyl)-4,4,4-trifluoro-butyryl]-oxazolidin-2-one (170 g) and THF (1.1 L) were added to a 5 L reaction flask. The mixture was cooled to −78° C. and a solution of KHMDS (0.5 M in toluene, 905 ml) was added over 30 minutes. The reaction mixture was aged at −78° C. for 30-45 min. Trisyl azide (131.2 g, 411 mmol) and THF (500 mL) were added to a separate 1 L flask and the mixture cooled to −78° C. Using a large cannula, the trisyl azide solution was added over 2 minutes to the solution of starting material and KHMDS. The reaction was stirred at −70° C. for 2 minutes and acetic acid (114 g) was added in one portion. The reaction was warmed to room temperature over 30 minutes and water (680 mL) was added. The organic layer was separated, THF was removed in vacuo and 2 L MTBE was added. The organic layer was washed with 0.5 N HCl (750 mL), then 1M K₂CO₃ (750 mL), giving 3 distinct layers. The bottom aqueous layer was removed, the top two layers were washed with 1M K₂CO₃ (750 mL) and the bottom two layers were removed. The top layer was washed with K₂CO₃ (750 mL) and brine (750 mL). The solvent was removed in vacuo and residue was used without purification in the following reduction step.

### Method 2:

(S)-4-Benzyl-3-[(R)-3-(3,5-difluoro-phenyl)-4,4,4-trifluoro-butyryl]-oxazolidin-2-one (10 g, 24.19 mmol) and THF (25 mL) were added to a 250 mL reaction flask. The mixture was cooled to 40° C. and a solution of KHMDS (29 mL of a 0.91 M in THF) was added over 10 minutes. The solution was aged at −40° C. for 30-45 min. A solution of trisyl azide (25.73 g of a 30 wt % solution in toluene) was cooled to −78° C. and added via a cannula to the solution of starting material and KHMDS while the internal temperature was maintained below −34° C. The reaction was stirred at −40° C. for 2 minutes and acetic acid (6.7 g) was added in one portion. Water (50 mL) was added and the mixture is warmed to ambient temperature. The water layer was cut and the THF is washed with water (50 mL) and brine (2×50 mL). The final organic layer was concentrated to a final volume of about 10 mL.

### Example 7

### Preparation of 3-[(S)-2-Amino-(R)-3-(3,5-difluoro-phenyl)-4,4,4-trifluoro-butyryl]-(S)-4-benzyl-oxazolidin-2-one hydrochloride

### Method 1:

The residue from the previous step (167 g) was combined in EtOH (1 L) and transferred to a 2 L PARR® shaker bottle. Concentrated HCl (117 g) was added, followed by addition of the palladium on carbon catalyst (70 g of 50% water-wet catalyst (10% palladium on carbon)). The reaction bottle was setup in a PARR® Shaker and hydrogenated under 50 psi (3.4 bar, 0.34 Mpa) H₂ for six hours (or until the reaction is complete as judged by HPLC). After the reaction completed, methanol (800 mL) was added and the mixture stirred for 30 minutes. The solution was filtered through a sintered glass funnel with a bed of the CELIT® reagent. The solution was concentrated in vacuo and ethanol (600 mL) was added. The mixture was concentrated to about 300 mL total volume and this volume was maintained as heptane (150 mL) was added portion-wise. The mixture was concentrated to a final total volume of about 250 mL and the slurry cooled to 0° C. for 45 minutes and filtered. The cake was washed with 100 mL cold 1:1 EtOH:Heptane. The product was isolated as a white solid (146 g; 77% two steps).

### Method 2:

The residue from the azidation of step F (10 g, 22 mmol) was dissolved in THF (about 20 mL) and concentrated HCl (6.5 g, 66 mmol) was added. Triphenylphosphine (23.67 g, 45 mmol) was dissolved in THF (20 mL) and the resulting solution was added dropwise over 1 hours to the mixture of starting material and HCl. After the addition completed, the reaction was stirred for 10 minutes at room temperature, the solvent was removed via concentration under vacuum, and EtOH (50 mL) was added. The solution was dried by evaporation from EtOH and heptane was added. The product that precipitated was collected by filtration and dried on the filter to afford the product (6.55 g, 58% over two steps).

### Example 8

### Preparation of (S)-2-Amino-(R)-3-(3,5-difluoro-phenyl)-4,4,4-trifluoro-butan-1-ol hydrochloride

THF (525 mL) and 3-[(S)-2-Amino-(R)-3-(3,5-difluoro-phenyl)-4,4,4-trifluoro-butyryl]-(S)-4-benzyl-oxazolidin-2-one hydrochloride (104.5 g) were added to a 3 L reactor and the slurry/suspension was cooled to −5 to 0° C. LiBH₄ (258 mL of 1.0 M THF solution) was dropwise added, while maintaining the internal temperature to less than 5° C. The solution was stirred at 5° C. for 15 minutes and then the solution warmed to 20° C. After disappearance of the starting material (monitored by HPLC), the solution was cooled to −5 to 0° C. MeOH (160 mL) was added to the solution, while maintaining the internal temperature below 10° C. 6 N HCl (248 mL) was added to the solution while maintaining the internal temperature below 10° C. The reaction mixture was warmed to 20-30° C. and the mixture stirred for 30-60 minutes, while monitoring decomposition of the intermediate borane complex via HPLC decomposition. The organic solutions were removed via vacuum distillation and the slurry cooled to 20° C. 2 N HCl (200 mL), water (600 mL) and methylene chloride (500 mL) were added and stirred for 15-30 minutes. The mixture was allowed to settle and the layers were separated. The upper aqueous layer was extracted with DCM (500 mL), the organic layers were combined, and potassium carbonate (120 g) was added in portions. The aqueous layer was extracted with MTBE (2×500 mL), NaCl (45 g) was added to the aqueous layer, and the aqueous layer extracted with MTBE (500 mL). The organic layers were combined and concentrated under vacuum to afford a crude oil. Diethyl ether (100 mL) and MeOH (15 g) were added, the solution cooled to 10-15° C., and 1.0 M HCl in diethyl ether (440 mL) was added. The slurry was stirred at 20-25° C. for 1 hour, the slurry cooled to 0° C. over 30 minutes, and the solution held for 2-3 hours. The solid was collected by filtration and the solid was washed with ether/heptane (1/9; 100 mL). The solids were dried to provide 54.3 g of product. (83% yield, 95.5% LC Purity).

### Example 9

### Preparation of 5-Chloro-thiophene-2-sulfonic acid [(1S,2R)-2-(3,5-difluoro-phenyl)-3,3,3-trifluoro-1-hydroxymethyl-propyl]-amide

### Method 1:

(S)-2-Amino-(R)-3-(3,5-difluoro-phenyl)-4,4,4-trifluoro-butan-1-ol hydrochloride (222 g) was added to a 5-L reaction flask. Isopropyl acetate (1.3 L) was added and the mixture stirred at room temperature to dissolve the solids. Ethyldiisopropylamine (246 g) was added and the mixture heated to 45-50° C. 5-chlorothiophene-2-sulfonyl chloride (157 g) was added via an addition funnel dropwise at 50° C. The reaction mixture was heated at 50° C. until complete by HPLC (about 1.5 hours) and the reaction mixture cooled to room temperature. Water (1.5 L) was added, the mixture stirred for 10 minutes, and the two layers separated. The organic layer was concentration under vacuum and co-evaporated with heptane (1.5 L). Heptane (1 L) was added, adjusted to an ambient temperature and the solid filtered. The product was washed with heptane, dried in a vacuum oven for 16 hours, and the product (240 g, 72.4% yield. 90% AN HPLC purity) collected.

### Method 2:

(S)-2-Amino-(R)-3-(3,5-difluoro-phenyl)-4,4,4-trifluoro-butan-1-ol hydrochloride (44.83 g) and 4-dimethylaminopyridine (1.02 g) were added to a 1-L reaction flask. Methylenechloride (400 mL) was added and the mixture was cooled to 0 to 5° C. N-methylmorpholine (32.7 g) was added and the mixture maintained at 0 to 10° C. 5-chlorothiophene-2-sulfonyl chloride (34.4 g) was added via an addition funnel dropwise at 0 to 10° C. The reaction mixture was held at 5° C. until complete by HPLC (about 16 hours). A solution of concentrated hydrochloric acid (15 g) and water (219 g) was added and the mixture was adjusted to 25 to 30° C. The mixture was stirred at 25 to 30° C. for 10 min., and the two layers separated. The organic layer was washed with a solution of concentrated hydrochloric acid (15 g) and water (219 g) at 25 to 30° C. for 10 min., and the two layers separated. The organic layer was washed with water (250 mL) at 25 to 30° C. for 10 minutes, and the two layers separated. The organic layer was concentrated at atmospheric pressure to approximately half its original volume, and heptane (400 mL) was added. The mixture was cooled to 0 to 5° C. and the solid filtered. The product was washed with a mixture of methylene chloride and heptane (1:4 v/v, 250 mL total volume) then dried on the filter to provide the product (55 g, 83% yield. 94% AN HPLC purity).

### Example 10

### Purification of 5-Chloro-thiophene-2-sulfonic acid [(1S,2R)-2-(3,5-difluoro-phenyl)-3,3,3-trifluoro-1-hydroxymethyl-propyl]-amide

The crude product (312 g) of step I was added to a 5-L reaction flask. 2-propanol (1.0 L) was added and the mixture heated to 40° C. until the solids dissolved. The solution was filtered through the CELITE® reagent (30 g-8.5 cm diameter×1.5 cm height) and charcoal pad, and the flask was rinsed and filtered with 2-propanol (100 mL). The filtrate and wash was combined and heated to 40° C. Water (540 mL) was added slowly at 40° C. The temperature was decreased slowly and the mixture seeded with product. The product precipitated at about 25° C. Water (1.2 L) was slowly added and the mixture stirred overnight at ambient temperature. Water (200 mL) was added and cooled to 7 to 10° C. for 3 hours. The solids were filtered and the cake washed with 1:6 IPA:H₂O (v/v) (250 mL). The product was dried in a vacuum oven for 16 hours and purified product collected (295.6 g, 95% recovery, 98.7% AN HPLC purity); mp 126° C.

### Example 11

### Analysis of 5-Chloro-thiophene-2-sulfonic acid [(1S,2R)-2-(3,5-difluoro-phenyl)-3,3,3-trifluoro-1-hydroxymethyl-propyl]-amide

A sample of 5-Chloro-thiophene-2-sulfonic acid [(1S,2R)-2-(3,5-difluoro-phenyl)-3,3,3-trifluoro-1-hydroxymethyl-propyl]-amide prepared as described herein and purified according to Example 10 was analyzed using powder X-ray diffraction.

X-Ray diffraction data was acquired using a D8 ADVANCE® X-ray powder diffractometer (Bruker) having the following parameters and the X-ray diffraction pattern was obtained. See, FIG. 1.

| | |
|---|---|
| voltage: | 40 kV; |
| current: | 40.0 mA; |
| scan range (2θ): | 5 to 35°; |
| scan step size: | 0.01°; |
| total scan time: | 33 minutes; |
| detector: | VANTEC ™ detector; and |
| antiscattering slit: | 1 mm. |

All publications cited in this specification are incorporated herein by reference. While the invention has been described with reference to particular embodiments, it will be appreciated that modifications can be made without departing from the spirit of the invention. Such modifications are intended to fall within the scope of the appended claims.

What is claimed is:

1. A method for preparing a sulfonamide compound of the structure: 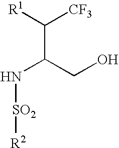
wherein:
R¹ is selected from the group consisting of C₁ to C₁₀ alkyl, C₁ to C₁₀ substituted alkyl, C₃ to C₈ cycloalkyl, C₃ to C₈ substituted cycloalkyl, aryl, substituted aryl, heteroaryl, and substituted heteroaryl;
R² is aryl, substituted aryl, heteroaryl, or substituted heteroaryl; said method comprising:
(a) reacting trifluoroacetic anhydride, morpholine, and a proton scavenger to form N-trifluoroacetylmorpholine;
(b) reacting N-trifluoroacetylmorpholine with R¹MgX or R¹Li to form R¹C(O)CF₃;
wherein, X is Br, Cl, or I;
(c) condensing R¹C(O)CF₃ with a phosphonoacetate reagent;
(d) hydrolyzing the product of step (c) to form R¹C(CF₃)═CHCO₂H;
(e) converting R¹C(CF₃)═CHCO₂H to a mixed anhydride;
(f) reacting said mixed anhydride with a chiral auxiliary compound;
(g) reducing the product of step (f);
(h) converting the product of step (g) to an azide;
(i) converting said azide to an amine salt;
(j) reducing said amine hydrochloride salt to an amino alcohol salt of the structure: 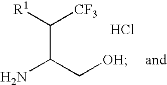
(k) reacting said amino alcohol salt with R²SO₂LG;
wherein LG is a leaving group.

2. The method according to claim 1, wherein said phosphonoacetate reagent is a trialkyl phosphonoacetate, triaryl phosphonoacetate, dialkylaryl phosphonoacetate, diarylalkylphosphonoacetate, or fluoroalkyl phosphonoacetate.

3. The method according to claim 2, wherein said phosphonoacetate reagent is triethylphosphonoacetate.

4. The method according to claim 1, wherein said chiral auxiliary is:
(i) an oxazolidinone of the structure: 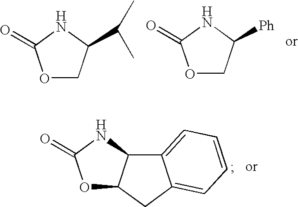
(ii) an imidazolidinone of the structure:

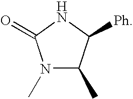

5. The method according to claim 1, wherein R² is of structure (i) or (ii):
(i) 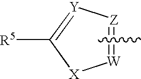
wherein:
W, Y and Z are independently selected from the group consisting of C, CR⁶ and N, wherein at least one of W, Y or Z is C;
X is selected from the group consisting of O, S, SO₂, and NR⁷;
R⁵ is selected from the group consisting of H, halogen, and CF₃;
R⁶ is selected from the group consisting of H, halogen, C₁ to C₆ alkyl, and substituted C₁ to C₆ alkyl;
R⁷ is selected from the group consisting of H, C₁ to C₆ alkyl, and C₃ to C₈ cycloalkyl; or
(ii) 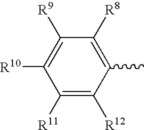
wherein:
R⁸, R⁹, R¹⁰, R¹¹, and R¹² are independently selected from the group consisting of H, halogen, C₁ to C₆ alkoxy, substituted C₁ to C₆ alkoxy, NO₂, C₁ to C₆ alkyl, and substituted C₁ to C₆ alkyl; or
R⁸ and R⁹; R⁹ and R¹⁰; R¹¹ and R¹²; or R¹⁰ and R¹¹ are fused to form:
(i) a carbon-based saturated ring containing 3 to 8 carbon atoms;
(ii) a carbon-based unsaturated ring containing 3 to 8 carbon atoms; or
(iii) a heterocyclic ring containing 1 to 3 heteroatoms selected from the group consisting of O, N, and S in the backbone of the ring;
wherein rings (i) to (iii) may be substituted by 1 to 3 substituents including C₁ to C₆ alkyl or substituted C₁ to C₆ alkyl.

6. The method according to claim 1, wherein the product of step (b) is:

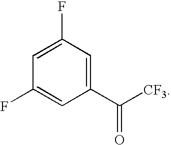

7. The method according to claim 1, wherein the product of step (c) is of the structure:

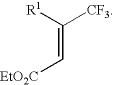

8. The method according to claim 7, wherein the product of step (c) is:

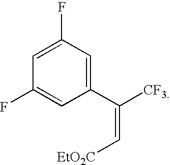

9. The method according to claim 1, wherein the product of step (d) is:

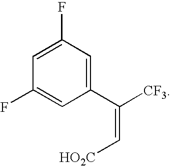

10. The method according to claim 1, wherein the product of step (e) is of the structure:

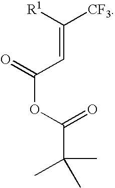

11. The method according to claim 10, wherein the product of step (e) is:

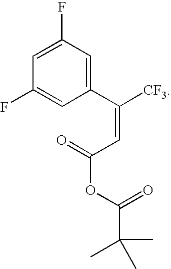

12. The method according to claim 1, wherein said unsaturated oxazolidinone is of the structure:

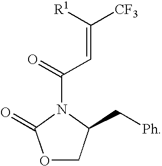

13. The method according to claim 12, wherein said unsaturated oxazolidinone is:

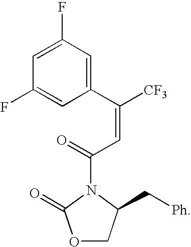

14. The method according to claim 1, wherein said saturated oxazolidinone is of the structure:

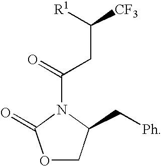

15. The method according to claim 14, wherein said saturated oxazolidinone is:

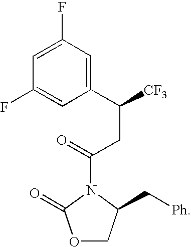

16. The method according to claim 1, wherein azide is of the structure:

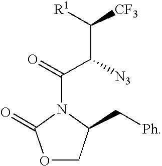

17. The method according to claim 16, wherein said azide is:

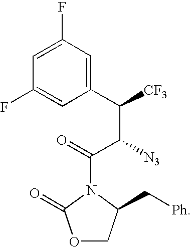

18. The method according to claim 1, wherein said amine salt is of the structure:

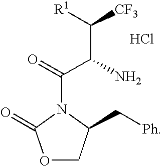

19. The method according to claim 18, wherein said amide hydrochloride salt is:

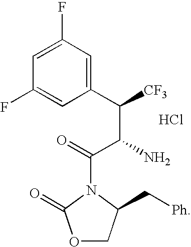

20. The method according to claim 1, wherein the product of step (k) is:

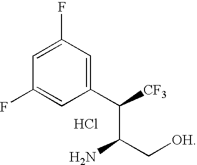

21. The method according to claim 1, wherein said sulfonamide compound is:

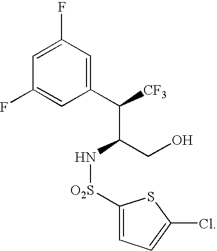

22. The method according to claim 1, wherein said R²C(CF₃)═CHCO₂H is isolated by adjusting the pH of the product of step (d) to less than about 3 and extracting said R²C(CF₃)═CHCO₂H.

23. The method according to claim 21 wherein 5-chloro-thiophene-2-sulfonic acid [(1S,2R)-2-(3,5-difluoro-phenyl)-3,3,3-trifluoro-1-hydroxymethyl-propyl]-amide has an X-ray diffraction peak pattern comprising a peak at 20 of about 6.5°.

24. The method according to claim 23, wherein said X-ray diffraction pattern further comprises one or more peaks at 20 of about 14.9°±0.3°, 22.1°±0.3°, 18.3°±0.3°, 19.6°±0.3°, 24.4°±0.3°, or 26.2°±0.3°.

25. A method for preparing (E)-3-(3,5-Difluoro-phenyl)-4,4,4-trifluoro-but-2-enoic acid, comprising:
(a) reacting N-trifluoroacetylmorpholine with bromo-3,5-difluorobenzene to form 1-(3,5-difluoro-phenyl)-2,2,2-trifluoro-ethanone;
(b) reacting 1=(3,5-difluoro-phenyl)-2,2,2-trifluoro-ethanone with triethylphosphonoacetate to form 3-(3,5-difluoro-phenyl)-4,4,4-trifluoro-but-2-enoic acid ethyl ester; and
(c) hydrolyzing 3-(3,5-difluoro-phenyl)-4,4,4-trifluoro-but-2-enoic acid ethyl ester.

26. A method for preparing (S)-4-Benzyl-3-[3-(3,5-difluoro-phenyl)-4,4,4-trifluoro-but-2-enoyl]-oxazolidin-2-one, comprising steps (a)-(c) of the method of claim 25 and further comprising:
(d) converting E-3-(3,5-difluoro-phenyl)-4,4,4-trifluoro-but-2-enoic acid to a mixed anhydride; and
(e) reacting said mixed anhydride with 4-(S)-benzyl-oxazolidin-2-one or a salt thereof to form 4-benzyl-3-[3-(3,5-difluoro-phenyl)-4,4,4-trifluoro-but-2-enoyl]-oxazolidin-2-one.

27. A method for preparing (S)-4-Benzyl-3-[(R)-3-(3,5-difluoro-phenyl)-4,4,4-trifluoro-butyryl]-oxazolidin-2-one, comprising steps (a)-(e) of the method of claim 26 and further comprising:
(f) reducing 4-benzyl-3-[3-(3,5-difluoro-phenyl)-4,4,4-trifluoro-but-2-enoyl]-oxazolidin-2-one.

28. A method for preparing 3-[(S)-2-Azido-(R)-3-(3,5-difluoro-phenyl)-4,4,4-trifluoro-butyryl]-(S)-4-benzyl-oxazolidin-2-one, comprising steps (a)-(f) of claim 27 and further comprising:
(g) reacting (S)-4-benzyl-3-[(R)-3-(3,5-difluoro-phenyl)-4,4,4-trifluoro-butyryl]-oxazolidin-2-one with trisyl azide.

29. A method for preparing 3-[(S)-2-Amino-(R)-3-(3,5-difluoro-phenyl)-4,4,4-trifluoro-butyryl]-(S)-4-benzyl-oxazolidin-2-one hydrochloride, comprising steps (a)-(g) of the method of claim 27 and further comprising:
(h) reacting 3-[(S)-2-azido-(R)-3-(3,5-difluoro-phenyl)-4,4,4-trifluoro-butyryl]-(S)-4-benzyl-oxazolidin-2-one with hydrogen, a dry catalyst, and hydrochloric acid.

30. A method for preparing (S)-2-Amino-(R)-3-(3,5-difluoro-phenyl)-4,4,4-trifluoro-butan-1-ol hydrochloride, comprising steps (a)-(h) of the method of claim 29 and further comprising:
(i) reducing 3-[(S)-2-amino-(R)-3-(3,5-difluoro-phenyl)-4,4,4-trifluoro-butyryl]-(S)-4-benzyl-oxazolidin-2-one hydrochloride.

31. A method for preparing 5-chloro-thiophene-2-sulfonic acid [(1S,2R)-2-(3,5-difluoro-phenyl)-3,3,3-trifluoro-1-hydroxymethyl-propyl]-amide, comprising steps (a)-(i) of claim 30 and further comprising:
(j) reacting (S)-2-amino-(R)-3-(3,5-difluoro-phenyl)-4,4,4-trifluoro-butan-1-ol hydrochloride with 5-chlorothiophene-2-sulfonyl chloride.

32. A compound which is N-trifluoroacetylmorpholine.

33. A method for preparing the compound of claim 32, comprising reacting trifluoroacetic anhydride, morpholine, and triethylamine.

34. A process for trifluoroacetylating a compound using a compound of claim 32.

35. A compound of the structure: [chemical structure]
